(19) United States
(12) Patent Application Publication
(10) Pub. No.: US 2024/0316298 A1
(43) Pub. Date: Sep. 26, 2024

(54) DEVICE FOR CALIBRATING THE SENSORS OF AN NO DELIVERY APPARATUS

(71) Applicant: INOSYSTEMS, ANTONY (FR)

(72) Inventors: Thierry BOULANGER, Newark, DE (US); Frederic MARCHAL, Antony (FR); Akshar PATEL, Newark, DE (US)

(21) Appl. No.: 18/610,366

(22) Filed: Mar. 20, 2024

(30) Foreign Application Priority Data
Mar. 21, 2023 (FR) 2302598

(51) Int. Cl. A61M 16/00

(52) U.S. Cl. CPC A61M 16/0003; A61M 2205/3327; A61M 2205/3337

(57) ABSTRACT

The invention relates to a calibration device (2) for calibrating the measuring means (120-122) of an NO delivery apparatus (1), comprising a gas intake line (22) comprising, arranged in series, a valve, preferably manual (23) and actuatable by a user, for controlling the circulation of gas in the gas intake line (22); a precision regulator (24) configured to deliver a predefined fixed pressure in the gas intake line (22); a calibrated orifice device (25), and a venturi device (26). The measuring means of the NO delivery apparatus comprise an NO sensor, an NO2 sensor and an O2 sensor.

### CROSS-REFERENCE TO RELATED APPLICATION

This patent application claims priority to FR 2302598, filed Mar. 21, 2023, and the entire contents of which are incorporated herein by reference.

### FIELD OF THE INVENTION

The invention relates to a calibration device for calibrating the measuring means of an NO delivery apparatus, in particular the NO, O2 and NO2 sensors of said NO delivery apparatus.

### BACKGROUND

Inhaled nitric oxide, or NOi, is a gaseous medicament commonly used to treat patients suffering from acute pulmonary arterial hypertension, in particular pulmonary vasoconstrictions in adults or children, including the newborn (PPHN), as described for example in EP-A-560928 or EP-A-1516639.

To implement inhaled NO therapy, a gas supply installation, also called an NO administration installation, is conventionally used, comprising an NO delivery apparatus, a medical ventilator, that is to say a respiratory assistance apparatus, and a patient circuit.

The NO delivery apparatus makes it possible to inject an NO-based gas mixture, typically an NO/nitrogen mixture, into the patient circuit supplied, moreover, with a gas stream containing oxygen (about 21% vol.), such as air or an oxygen/nitrogen mixture, supplied by the medical ventilator.

The patient circuit generally comprises one or more flexible conduits fluidically connected to a respiratory interface, such as a tracheal intubation tube or the like, serving to deliver to the patient to be treated a given dose of NO, i.e. a dosage.

Such a gas supply installation is described, for example, in EP3821929. This type of installation is used in a hospital environment to administer treatment with NOi and thus care for patients who need to inhale NO in order to treat their pulmonary arterial hypertension.

In order to ensure that the gas mixture administered to the patient contains the desired proportions of NO and oxygen but, conversely, contains little or no NO2, it is advisable to regularly take gaseous samples from the patient circuit, typically near the respiratory interface, and to analyse them.

To do this, the NO delivery apparatus is fluidically connected to the patient circuit via a gas sampling line, in order to allow sampling of a portion of the gas flowing therein, typically about 100 to 300 ml/min, in order to analyse it and to check whether the gas contents are in accordance with the desired values, in particular of NO, NO2 and O2.

EP2522384 proposes a gas analyser arranged on board the NO delivery apparatus, serving to measure the contents of NO, NO2 and O2 in the gas samples that have been taken. The means of measuring concentration use sensors of the electrochemical type.

However, sensors of this type suffer from drift over the course of time and require periodic calibration. A “zero” calibration of the sensors requires a reference gas mixture free of NO and NO2 in order to determine their nominal quiescent response, in particular by sampling ambient air. Moreover, the generation of the high calibration point, for example at 40 ppmv of NO (gain), is effected via an all-or-nothing solenoid valve connecting the NO source (i.e. mixture of 800 ppmv of NO diluted in N2) to the gas analyser.

This solution is not ideal, however, since the fact of using a solenoid valve between the NO source and the gas analyser requires a profound modification to the architecture of the NO delivery apparatus, in particular entailing significant changes to its electronic and mechanical components. It is therefore not possible or easy to integrate this solution in existing apparatuses, that is to say those already in service. Moreover, such a solenoid valve, even when closed, may be subject to small leaks, and “leaked” NO may mix with the gas to be analysed and then cause major interference at the sensors.

Furthermore, EP2581103 describes a method for calibrating an NO supply apparatus, US2015/320951 teaches a method for predicting when an NO cylinder feeding an NO supply apparatus will be empty, and CN104857607 proposes an oxygen concentration calibration device.

Proceeding from this, a problem is to allow effective periodic calibration of an NO delivery apparatus, autonomously and independently of the NO delivery apparatus, specifically on any kind of NO delivery apparatus, including those in the existing stock, preferably without risk of interference with the gas analyser of the NO delivery apparatus in question.

### SUMMARY

A solution of the invention relates to a calibration device for calibrating the measuring means of an NO delivery apparatus, said measuring means comprising an NO sensor, an NO2 sensor and an O2 sensor, comprising a gas intake line comprising, arranged in series:
a valve for controlling the circulation of gas in the gas intake line,
a precision regulator configured to deliver a predefined fixed pressure in the gas intake line,
a calibrated orifice device, and
a venturi device.

In addition, the venturi device comprises a main body defining an internal volume and comprising:
a gas inlet orifice fluidically connected to the calibrated orifice device in order to allow gas from the calibrated orifice device to enter the internal volume,
a gas intake orifice fluidically connected to the atmosphere in order to allow air to enter the internal volume,
an exhaust orifice fluidically connected to the atmosphere in order to allow some of the gas contained in the internal volume to be discharged to the atmosphere, and
an outlet port for supplying at least some of the gas contained in the internal volume to an NO delivery apparatus.

Depending on the embodiment considered, the calibration device of the invention can comprise one or more of the following features:
the valve is movable between at least:
   a closed position, i.e. valve closed, in which the circulation of gas in the gas intake line is prevented,
   and an open position, i.e. valve open, in which the circulation of gas in the gas intake line is permitted.
the valve is a manual valve actuatable by a user, i.e. actuatable between at least the two positions.
the precision regulator is configured to deliver a fixed pressure of between 350 and 700 mbar.
the calibrated orifice device comprises an outlet orifice with a diameter (non-zero) of less than 500 μm.
the gas intake line, the manual valve, the precision regulator, the calibrated orifice device and the venturi device are arranged in a common housing.
the gas intake orifice has a cross-sectional area of between 0.5 and 10 mm2.
it further comprises upstream connection means arranged at an inlet port of the gas intake line and configured to connect a flexible conduit thereto.
it further comprises downstream connection means arranged at the outlet port of the venturi device and configured to connect thereto a gas feed line of a gas analyser of an NO delivery apparatus.
the outlet diameter of the calibrated orifice and the diameter of the venturi intake orifice are dimensioned to obtain a constant ratio, between the flow rate leaving the calibrated orifice and the flow rate entering through the venturi intake orifice, of between 10 and 30.
the outlet diameter of the calibrated orifice and the diameter of the venturi intake orifice are dimensioned to obtain a constant ratio over a given pressure range of between 200 and 900 mb, for example.

The invention also relates to the use of a calibration device according to the invention, for calibrating the measuring means of an NO delivery apparatus, said measuring means comprising an NO sensor, an NO2 sensor and an O2 sensor.

Preferably, the calibration device is fluidically connected to one or more gas cylinders containing an NO/N2 mixture feeding the gas intake line of the calibration device, upstream of the manual valve.

Advantageously, the pressurized gas cylinder contains an NO/nitrogen mixture containing from 100 to 2000 ppmv of NO diluted in nitrogen (N2), typically between 200 and 1500 ppmv of NO, the remainder being nitrogen.

### BRIEF DESCRIPTION OF THE VIEWS OF THE DRAWINGS

The invention will now be better understood from the following detailed description, given as a non-limiting example, with reference to the appended figures, in which:

FIG. 1 shows schematically an embodiment of a calibration device according to the present invention;

FIG. 2 is a table of results illustrating tests carried out with the calibration device of FIG. 1;

FIG. 3 shows schematically an embodiment of the gas analyser of an NO delivery apparatus that is to be periodically calibrated;

FIG. 4 shows schematically the calibration device of FIG. 1 associated with the NO delivery apparatus of FIG. 3, during a calibration procedure;

FIG. 5 illustrates the performance of a pump forming part of the gas analyser of FIG. 3; and

FIG. 6 shows schematically an embodiment of an installation for supplying gas to a patient, comprising the NO delivery apparatus of FIG. 3 and a medical ventilator feeding a patient circuit.

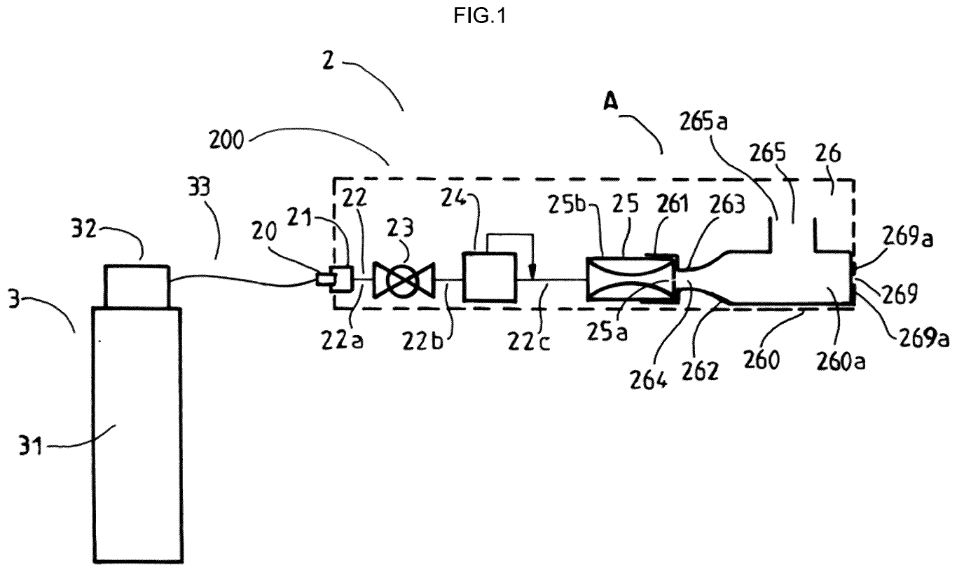

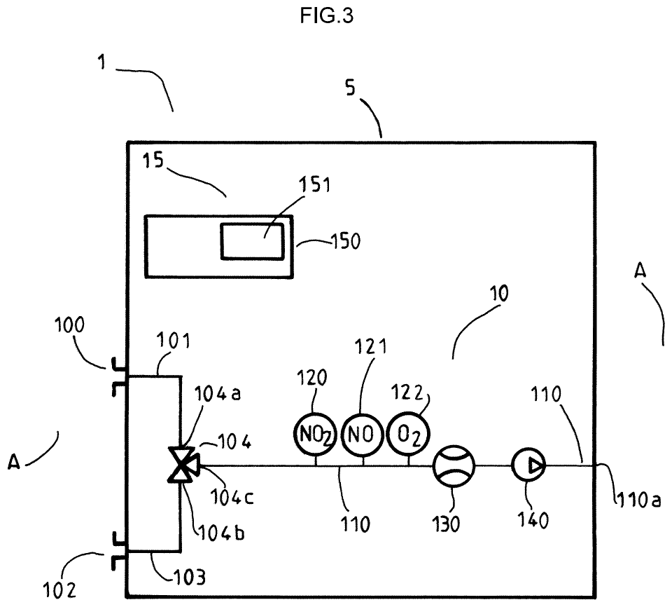

FIG. 1 shows schematically a calibration device 2, i.e. a calibration system or assembly, according to the invention, which can be used for calibrating the sensors of an NO delivery apparatus, such as that illustrated in FIG. 3, as set out in detail below.

### DETAILED DESCRIPTION

In FIG. 1, the calibration device 2 according to the invention is connected, at an inlet port 21, to a flexible conduit 33, which is itself fluidically connected to an NO source 3. The connection is made via upstream connection means 20 located at the inlet port 21, such as a connector or the like, preferably of the “quick-connect” type, which enables a user to connect and disconnect the connection hose simply and quickly.

Here, the NO source 3 is a pressurized gas cylinder 31 typically containing an NO/nitrogen mixture, preferably an NO/nitrogen mixture containing from 100 to 2000 ppmv of NO diluted in nitrogen, for example containing here about 800 ppmv of NO diluted in nitrogen (N2). The internal volume of the gas cylinder 31 is preferably between 2 and 20 litres (water equivalent). The NO/N2 mixture is stored therein at a pressure of at least 150 bar, preferably at least 180 bar, for example of the order of 200 bar or more, when it is full, that is to say before any withdrawal of gas.

The gas cylinder 31 is surmounted by a pressure-reducing valve 32 (or RDI) which makes it possible to lower the flow rate of gas and to reduce the pressure of the gas, coming from the cylinder 31, to a given fixed pressure of expansion, for example of between 3 and 7 bar, for example of the order of 4 or 5 bar.

The inlet port 21 of the calibration device 2 places the connection hose 33, fed with the gas coming from the cylinder 31, in fluidic communication with a gas intake line 22 on which there are arranged various components traversed successively by the gas, i.e. the NO/N2 mixture from the pressurized gas cylinder 31. The gas circulates therein in a direction of flow going from the inlet port 21 to the components 23, 24, 25, 26.

Thus, it will be seen that a valve 23, preferably manual, is arranged on the intake line 22 immediately downstream of the inlet port 21. The valve 23 is manoeuvrable, preferably by actuation on the part of the user, between an open position and a closed position, and vice versa, via a maneuvering member, such as a rotary knob (not shown), so as to control the circulation of gas in the intake line 22.

When the valve 23 is in the closed position, the upstream portion 22a of the intake line 22 situated before the valve 23 (i.e. upstream) is subjected to the expansion pressure of the regulator 31, whereas the intermediate portion 22b and the downstream portion 22c located after the valve 23 (i.e. downstream) are at atmospheric pressure.

Arranged downstream of the valve 23 is a precision regulator 24, which is adjusted to a predefined fixed position, that is to say which is configured to deliver a given fixed pressure, as described below, preferably of between 350 and 700 mbar, for example of the order of 500 mbar. It is possible, for example, to use the precision regulator sold by Beswick® under the commercial reference PRD.

Downstream of this regulator 24, a calibrated orifice device 25 is arranged on the intake line 22, here in the downstream portion 22c thereof. The diameter of this calibrated orifice device 25 is considered the outlet diameter, that is to say the diameter situated at its outlet 25a. The outlet diameter of the calibrated orifice device 25 is non-zero and preferably less than 500 μm.

It is possible, for example, to use the calibrated orifice device 25 sold by O'Keefe Control®, under the commercial reference BLP-2®, which has an outlet diameter of the order of 65 μm.

The calibrated orifice device 25 is mechanically coupled and fluidically connected to a venturi device 26 via a shoulder 261 of the venturi device 26 that is secured to the outer surface 25b of the calibrated orifice device 25, for example by screwing, force-fit or any other technique.

The venturi device 26 comprises a main body 260 defining an internal volume 260a. A neck 262 connects the shoulder 261 to the main body 260.

In addition, the venturi device 26, in particular the neck 262, comprises a gas intake orifice 263 in fluidic communication with the atmosphere. The intake orifice 263 can have different shapes, for example rectangular, circular or the like. Preferably, it has a cross-sectional area of between 0.5 and 10 mm2, for example of the order of about 5 mm2.

The internal volume of the neck 262 of the venturi device 26, which lies substantially between the outlet 25a of the calibrated orifice device 25 and the intake orifice 263, forms a venturi chamber 264.

The outlet 25a of the calibrated orifice device 25 is in fluidic communication with the internal volume 260a of the main body 260 of the venturi device 26, via the neck 262.

The main body 260 of the venturi device 26 moreover has an exhaust conduit 265 with an exhaust orifice 265a fluidically connected to the ambient atmosphere A, and an outlet orifice or port 269 which can be fluidically connected to an NO delivery apparatus 1, as explained below.

The gas intake orifice or port 263, the exhaust orifice 265a and the outlet port 269 are in fluidic communication with the internal volume 260a of the main body 260 of the venturi device 26.

All the elements forming the calibration system 2 according to the invention can be inserted in a rigid housing 200, shown only by broken lines, making it possible to ensure their mechanical integrity without affecting the performance of the assembly.

Hereinafter, it is considered that the valve 23 is manual and can be actuated by the user. It is called a “manual valve 23”.

When the user manoeuvres the manual valve 23 so as to move it to the open position, the pressure prevailing in the upstream portion 22a of the intake line 22 propagates downstream of the manual valve 23, as far as the precision regulator 24. The precision regulator 24 then generates a useful expansion pressure lower than the expansion pressure fixed by the regulator 32, preferably of between 350 and 700 mbar, for example of the order of 500 mbar. The useful expansion pressure then propagates in the downstream portion 22c of the intake line 22, upstream of the calibrated orifice 25.

Now, there is a relationship between the pressure upstream of the calibrated orifice device 25 and the flow rate leaving said calibrated orifice device 25 via its outlet diameter at its outlet 25a.

Because of the small dimension (i.e. <500 μm) of the outlet diameter of the outlet 25a, for example of the order of 65 μm, the calibrated orifice 25 generates a fluid at a low volume flow rate (for example of the order of 0.05 l/min) and at high velocity (for example of the order of 3 m/s) at the outlet 25a, which will create a negative pressure in the venturi chamber 264, which will itself create a negative pressure differential between the absolute pressure prevailing in the venturi chamber 264 and the absolute pressure, i.e. atmospheric pressure, of the ambient air A in fluidic communication with the venturi chamber 264 via the intake orifice 263.

The negative pressure differential created generates aspiration of a flow of air from the ambient atmosphere A via the inlet orifice 263. The aspirated air contains in particular 20.9 vol. % of O2 (i.e. about 21% of O2), negligible quantities of NO and NO2, i.e. of the order of 0.05 ppmv, and of course nitrogen and argon, or other negligible impurities such as water vapour.

The flow of air entering through the venturi intake orifice 263 mixes with the flow of NO/N2 mixture delivered through the calibrated orifice 25, i.e. via its outlet 25a. Here, the NO/N2 gas mixture from the gas cylinder 31 contains NO at a concentration of 800 ppmv, the remainder being nitrogen (N2).

By correctly dimensioning the outlet diameter (at the outlet 25a) of the calibrated orifice 25 and the venturi intake orifice 263, it is possible to obtain, over a given pressure range, that is to say of the pressure upstream of the calibrated orifice 25, i.e. in the downstream portion 22c of the intake line 22, a constant ratio between the flow rate leaving the calibrated orifice 25 and the flow rate entering through the venturi intake orifice 263. This ratio can be between 10 and 30, for example of the order of 19.

Thus, if the flow rate generated by the calibrated orifice 25 contains 800 ppmv of NO, and the flow rate entering through the venturi intake orifice 263 contains a negligible quantity of NO (e.g. about 0.05 ppmv), the mixture of the two gas flows contains an NO concentration of the order of 40 ppmv, i.e. because of the ratio of 19 here.

The gaseous mixture containing 40 ppm of NO then propagates in the internal volume 260a of the main body 260 of the venturi device 26 and then escapes to the ambient atmosphere A via the exhaust conduit 265 and the outlet port 269.

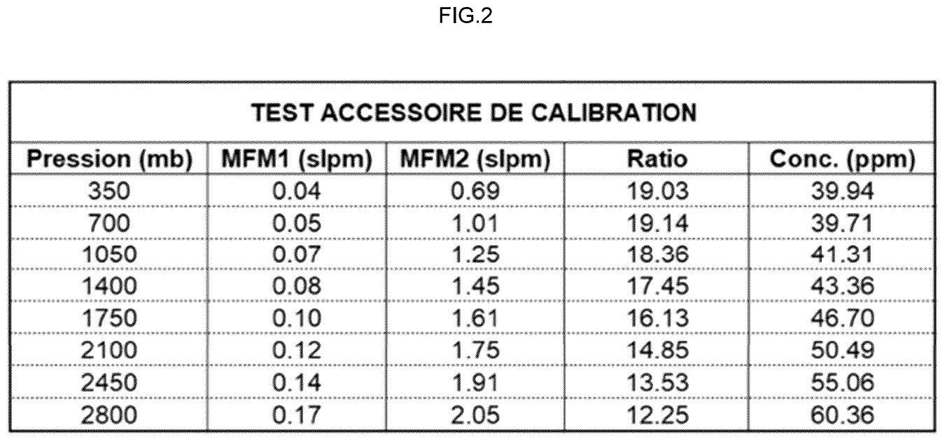

FIG. 2 is a table of results illustrating tests carried out with the calibration device 2 of FIG. 1, for useful expansion pressure values of between 350 and 2800 mbar.

For each useful expansion pressure, the flow rate MFM1 (in l/min) coming from the NO source 3 and passing through the calibrated orifice 25 and the flow rate MFM2 (in l/min) entering via the intake orifice 263 of the venturi device 26 were measured, and the values obtained made it possible to determine the ratio between the two flow rates (i.e. ratio between the flow rate entering via the intake orifice 263 and the flow rate generated by the calibrated orifice 25).

It can be seen that the ratio has a constant value of about 19, over a restricted pressure range, namely here between 350 and 700 mb. Beyond this, the ratio decreases as the useful expansion pressure increases. Thus, the ratio is only 12.25 for a pressure of 2800 mb. These results reflect the efficiency of any venturi device that reaches a maximum over a wide pressure range, is maintained over a narrow range within the wide pressure range, and then decreases as the pressure increases.

On this basis, in view of this change in the ratio between the two flow rates and for an NO/N2 mixture at 800 ppmv of NO from the NO source 3, as described above, the NO concentration resulting from the mixing of the two flows will be approximately 40 ppmv over the range (350 mbar-700 mbar) and will then gradually increase as the useful expansion pressure increases.

It is thus determined, for example, that the NO content is 43.36 ppmv at 1400 mbar and 60.36 ppmv at 2800 mbar, as shown in the table of FIG. 2.

The desired dilution ratio, for example 19 here, at its maximum efficiency for which stability is obtained over a given pressure range, can be obtained by specifically dimensioning the venturi device 26, in particular the intake orifice 263. This can be done, for example, via empirical dimensioning tests.

In view of the results in FIG. 2, it is preferable to adjust the precision regulator 24 to about 500 mb. In fact, the useful expansion pressure generated by the precision regulator 24 may vary or drift slightly over time. It is therefore judicious to fix the expansion value of the precision regulator 24 towards the middle of the narrow range 350-700 mbar in order to limit the risk of the ratio of 19 no longer being respected. In fact, if a drift of the precision regulator 24 occurs, for example if the pressure that it delivers drifts slightly above or below 500 mbar, the desired dilution ratio remains equal to about 19, that is to say to its maximum efficiency. This makes it possible to ensure a stability of the dilution ratio, even in the case of a slight variation or drift in the useful expansion pressure, and thus to maintain here an NO concentration of the order of 40 ppmv, after dilution by the ambient air fed through the intake orifice 263 of the venturi device 26.

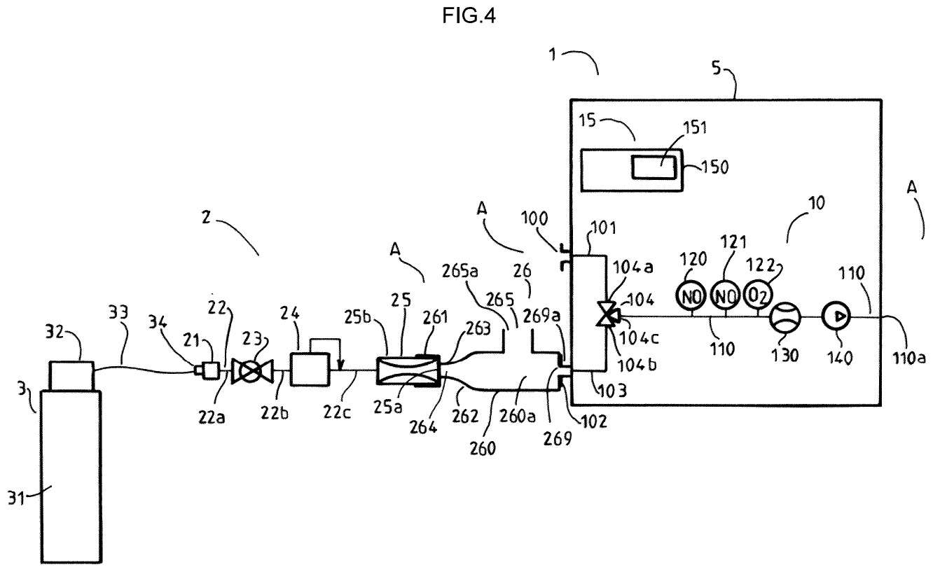

As is set out in detail below (cf. FIG. 4), the calibration device 2 of FIG. 1 can be used to calibrate the sensors of an NO delivery apparatus, such as that shown in FIG. 3, being part of an installation 1000 supplying gas to a patient and comprising said NO delivery apparatus 1 cooperating with a medical ventilator 300 and with a patient circuit 403, so as to inject NO into the patient circuit 403 which additionally conveys a respiratory gas containing at least about 21% vol. oxygen, typically air or an O2/N2 mixture, supplied by the medical ventilator 300.

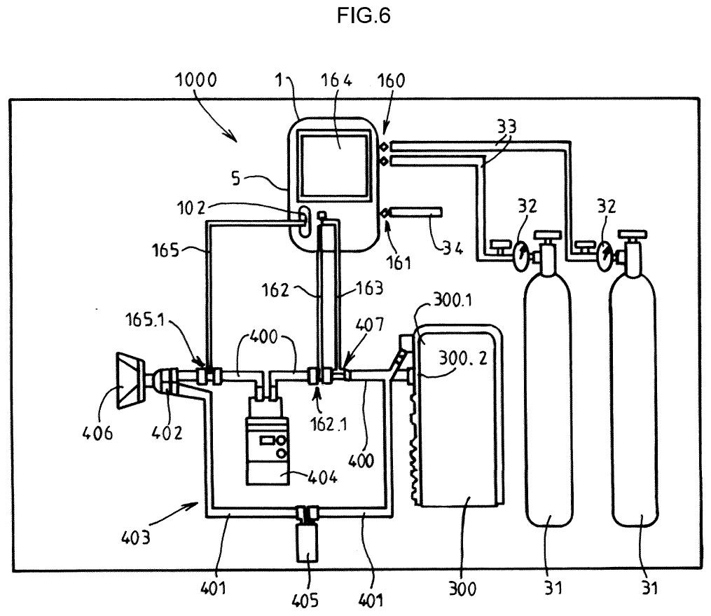

An embodiment of such a gas supply installation 1000 is illustrated in FIG. 6. Here, it comprises two pressurized gas cylinders 31, which each contain an NO/N2 gas mixture, namely here an NO/N2 gas mixture containing 800 ppm vol. of NO (remainder N2), and which feed NO/N2 mixture to the NO delivery apparatus 1.

The gas cylinders 31 are fluidically connected to the NO delivery apparatus 1 via gas feed lines 33, such as flexible hoses or conduits or the like, which may be equipped with devices for regulating and/or monitoring the gas pressure, such as gas regulator 32, pressure gauges, etc.

The gas feed lines 30 are connected to one or more gas inlets 160 of the NO delivery apparatus 1, which supply an internal gas passage serving to convey the gas within the NO delivery apparatus 1, that is to say in the housing 5 or the outer shell of the NO delivery apparatus 1.

The NO delivery apparatus 1 also comprises an oxygen inlet 161 fluidically connected, via an oxygen feed line 34 such as a flexible hose or the like, to an oxygen source, for example a pressurized oxygen cylinder or a hospital network, that is to say an oxygen supply line arranged in a hospital building.

The gas supply installation 1000 further comprises a medical ventilator 300, that is to say a respiratory assistance apparatus, which supplies a respiratory gas flow containing at least about 21% oxygen, such as air or an oxygen/nitrogen (N2/O2) mixture.

The medical ventilator 300 and the NO delivery apparatus 1 of the gas supply installation 1000 are in fluidic communication with a gas feed line 400, also called a patient circuit, serving to convey a gas flow to the patient, which is formed by mixing the flow from the medical ventilator 300 and the flow containing NO, i.e. the NO/N2 gas mixture, delivered by the NO delivery apparatus 1.

As has already been explained, the NO delivery apparatus delivers or injects the NO/N2 mixture, here at 800 ppmv of NO, into the gas feed line 400 via an injection conduit or line 162, so as to inject (at 162.1) the flow of NO/N2 into the flow of air or oxygen/nitrogen mixture delivered by the medical ventilator 300 and conveyed through the feed line 400.

The gas feed line 22 further comprises a gas humidifier 404 arranged downstream of the site (162.1) where NO is injected into the feed line 22. It makes it possible to humidify the flow of gas, e.g. NO/N2/air mixture, before it is inhaled by the patient to be treated by way of a respiratory interface 406 such as a tracheal intubation tube, a respiratory mask or the like.

A line 401 for recovering the gases exhaled by the patient is also provided. The gas feed line 400 and the exhaled-gas recovery line 401 are connected to a connection piece 402, preferably a Y-piece, and thus define a patient circuit 403. The gas feed line 400 forms the inspiratory branch of the patient circuit 403, while the recovery line 401 forms the expiratory branch of the patient circuit 403.

The gas feed line 400 is fluidically connected to an outlet port 300.1 of the medical ventilator so as to recover and convey the gas, typically air (or N2/O2 mixture containing about 21% O2) delivered by the medical ventilator 300, whereas the exhaled-gas recovery line 401 is fluidically connected to an inlet port 300.2 of the medical ventilator 300 so as to return to the medical ventilator 300 all or part of the flow of the gases exhaled by the patient.

The exhaled-gas recovery line 401 can comprise one or more optional components, for example a CO2 removal device 405, i.e. a CO2 trap, such as a hot container or the like, used to remove CO2 from the patient's exhaled gases, or a filter or the like. The recovery line 401 can be used by the ventilator 300 to detect a gas leak in the patient circuit 403.

A flow rate sensor 407, for example of the hot wire or pressure differential type, is arranged on the gas feed line 400, between the ventilator 300 and the humidifier 404, and is connected to the NO delivery apparatus 1 via a flow rate measuring line 163. This arrangement measures the flow rate of gas delivered by the ventilator 300, such as air or an N2/O2 mixture, and circulating in the feed line 400, upstream of the site 162.1 where the injection conduit or line 162 is connected, where the NO/N2/air mixture is made. This makes it possible to better regulate the delivery of the NO flow by the NO delivery apparatus 1.

As is set out in detail below, a gas sampling line 165 fluidically connects the gas feed line 400 to the NO delivery apparatus 1.

The gas sampling line 165 is fluidically connected (at 165.1) to the gas feed line 400, between the humidifier 404 and the junction piece 402, i.e. the Y-piece, typically in the immediate vicinity of the junction piece 402, and also to an inlet port 102 of the NO delivery apparatus 1, for example a port 102 carried by a connector, coupling or the like allowing the connection of the gas sampling line 165, such as a flexible hose or the like.

The gas sampling line 165 makes it possible to take gas samples from the gas feed line 400 of the patient circuit 403, for verification of their compliance, and to convey them to the NO delivery apparatus 1, where they are analysed in an internal gas analyser 10, as is set out in detail below.

In particular, it should be verified that their composition conforms with that of the desired NO/O2/N2 gas mixture to be administered to the patient, in particular in order to ensure that it does not contain excessive amounts of toxic NO2 species, that its oxygen content is not hypoxic, and that its NO content corresponds to the desired dosage.

This conformity check is conventionally carried out by dedicated measuring means, typically NO2, NO and O2 sensors, which themselves have to be calibrated periodically, for example every week.

Thus, FIG. 3 shows schematically an embodiment of the NO delivery apparatus 1 to which can be connected, as illustrated in FIG. 4, the calibration device 2 of FIG. 1 in order to enable calibration of the measuring means used to verify the conformity of the gas mixture, typically NO2, NO and O2 sensors.

This NO delivery apparatus 1 comprises, in a conventional manner, a rigid housing 13, for example made of polymer, through which an internal gas passage (not visible) passes, such as a gas conduit or the like, in order to convey the NO/N2 flow fed through the one or more gas feed lines 33, the latter being supplied by the NO/N2 mixture cylinders 31. The internal gas passage fluidically connects the gas inlet(s) 160 (cf. FIG. 6) of the NO delivery apparatus 1 to the injection line 162 in such a way as to convey the NO-based gas flow between them.

Conventionally, valve means (not shown), i.e. one or more valve devices, for example a plurality of solenoid valves arranged in parallel or one or more proportional (solenoid) valves, are arranged on the internal gas passage in order to control the gas flow which circulates therein in the direction of the injection line 162.

The valve means are controlled by control means 15, i.e. one or more control devices, arranged in the housing, typically an electronic card comprising one or more microprocessors, typically one or more microcontrollers, implementing one or more algorithms.

The control means 15 make it possible in particular to adjust or control the gas flow rate by controlling the valve means, typically to open or close said valve or valves, in order to obtain a gas flow rate determined and/or calculated by the control means 15 from a value set/fixed by the user, and as a function of the flow rate of gas, i.e. air, delivered by the ventilator 300 and measured by the flow rate sensor 407 arranged on the gas feed line 400 and connected to the NO delivery apparatus 1 by the flow rate measuring line 163, as is explained above. The flow rate measurements of the flow delivered by the ventilator 300 and circulating in the line 400 are supplied to the control means 150.

The internal gas passage can also comprise one or more flow meters (not shown) arranged upstream and/or downstream of the valve means, in order to determine the flow rate of NO-based gas circulating in the NO delivery apparatus 1. The flow meter can be of the differential-pressure type, the hot-wire type or some other type. It cooperates with the control means 150 in order to provide them, here again, with measurements of the flow rate of the NO/N2 flow.

Furthermore, the NO delivery apparatus 1 also comprises a graphical user interface (GUI) comprising a graphical display 174, preferably a touch screen, that is to say a touch panel, serving to display various information items or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare provider.

The control means 15 comprise, for example, an electronic control card 150 and a microprocessor-based control unit 151, typically a microcontroller or the like. The control means 15 make it possible to adjust or control all the electromechanical elements of the NO delivery apparatus 1. More precisely, the control card 150 preferably integrates the control unit 151 and is configured to control and also to analyse the signals coming from the various components of the NO delivery apparatus 1, such as the pump, sensors, etc.

The electrical supply to the NO delivery apparatus 1, in particular to the components requiring electrical current in order to operate, such as the control means 15, the graphical display 164, etc., is provided conventionally by an electrical current source and/or electrical supply means (not shown), for example a connection to the mains current (110/220V), such as an electrical cord and connection socket, and/or one or more electric batteries, preferably rechargeable, and/or a current transformer.

Furthermore, as has already been mentioned, the NO delivery apparatus 1 comprises an internal gas analyser 10, which is used during the calibration procedures. In the embodiment in FIG. 3, all or some of the elements of the gas analyser 10 can be arranged in the housing 5, which is made of polymer, for example, and which forms the external envelope of the NO delivery apparatus 1.

More precisely, the gas analyser 10 comprises a first inlet port 100 and a second inlet port 102, which are typically located outside the housing 5 of the NO delivery apparatus, making it possible to feed an analysis line 110 of the analyser 10 with gas, where:
the first inlet port 100 is fluidically connected to a first upstream port 104a of a 3:2 valve 104, via a first line 101; and
the second inlet port 102 is fluidically connected to a second upstream port 104b of the solenoid valve 104, via a second line 102.

The 3:2 valve 104, preferably a solenoid valve, further comprises a downstream port 104c fluidically connected to the analysis line 110, which comprises measuring means 120-122. The analysis line 110 also terminates at an outlet port 110a fluidically connected to the ambient atmosphere A.

This type of 3:2 (solenoid) valve 104 is commercially available, for example from the company IMI FAS®.

The control means 15 control the 3:2 (solenoid) valve 104 in such a way as to produce, as a function of a configuration determined by the control means 15, a fluidic connection between the first upstream port 104a, or alternatively the second upstream port 104b, and the downstream port 104c, hence with the analysis line 110.

For its part, the analysis line 110 comprises NO2 measuring means 120, such as an NO2 sensor, NO measuring means 121, such as an NO sensor 121, oxygen measuring means 122, such as an O2 sensor, and flow rate measurement means 130, such as a flow rate sensor. The NO2 measuring means 120, NO measuring means 121 and O2 measuring means 122 are of the electrochemical type. Such sensors are available from Honeywell.

Furthermore, the flow rate measurement means 130 determining the flow rate circulating in the analysis line are or preferably comprise a mass sensor, for example available from Sensirion®.

In addition, the analysis line 110 comprises a gas aspiration device 140, such as a pump or the like, preferably a diaphragm pump, making it possible to circulate a flow of gas in the analysis line 110, as is explained below. A pump that can be used is available from Parker® or Thomas®.

The control means 15 are configured to recover and process, i.e. analyse, the signals coming from the various sensors 120-121, 130 of the gas analyser 10, and to act in response to these signals, as is explained below, in particular to calibrate the sensors.

Thus, FIG. 4 shows schematically an association of the calibration device 2 of FIG. 1 of the invention with the NO delivery apparatus 1 of FIG. 3, so as to enable calibration of the NO2 measuring means 120-122, that is to say of the NO2, NO and O2 sensors.

To initiate a procedure of calibrating the NO2 measuring means 120-122, the user first fluidically connects the downstream connection means 269a, located at the outlet port 269 of the venturi device 26, to the second inlet port 102 of the gas analyser 10, for example by screwing, by interlocking or by any other type of connection capable of providing a fluidic connection between the internal volume 260a of the main body 260 of the venturi device and a gas feed line, i.e. referred to hereinbelow as the second line 103, of the 10 gas analyser of the NO delivery apparatus 1 that is to be calibrated.

The manual valve 23 is left or placed in the closed position, that is to say is closed, so that no pressurized gas can flow towards the intermediate portion 22b and the downstream portion 22c of the intake line 22.

The user then instructs the control means 15, for example via the GUI of the NO delivery apparatus 1, to start a sequence of calibration of the NO2 measuring means 120-122, i.e. the sensors. The control means 15 then control the solenoid valve 104 to place its downstream port 104c in fluidic communication with the first upstream port 104a, and furthermore the aspiration means 140, typically a pump, to aspirate ambient air A at a constant flow rate via the first inlet port 100 into the first line 101 and then the gas analysis line 110 respectively, before discharging this air to the ambient atmosphere A via the outlet port 110a.

The flow rate of air circulating in the gas analysis line 110 is kept constant, for example equal to approximately 250 ml/min, by the control means 15 thanks to the flow rate measurements performed by the flow rate sensor 130, which measurements are sent to the control means 15 and processed within these. The control means 15 therefore continuously adjust the control of the aspiration means 140 in order to obtain the desired target flow rate with respect to the measurements carried out.

In all cases, the gas flow circulating in the gas analysis line 110 is ambient air, with known and substantially constant concentrations of NO, NO2 and O2, namely an O2 concentration of the order of 20.9% and negligible concentrations, i.e. almost zero, of NO and NO2 (i.e. <0.05 ppmv).

Thus, it is possible to carry out a “zero” calibration of the NO2 sensor 120 and NO sensor 121, and a “complete” calibration of the O2 sensor 122 (i.e. 20.9% O2).

The user then opens the manual valve 23 in order to supply NO-based gas mixture, i.e. from the NO source 3 (i.e. 800 ppm NO here), to the intermediate and downstream portions 22b, 22c.

The downstream portion 22c is at the useful expansion pressure, because of the adjustment of the precision regulator 24, and it follows that, as has already been explained, a mixture here containing 40 ppmv of NO fills the internal volume 260a of the main body 260 of the venturi device 26.

At this stage, the solenoid valve 104 still fluidically connects its first upstream port 104a to its downstream port 104c, such that the second line 103 is isolated from the gas analysis line 110, i.e. the second upstream port 104b of said solenoid valve 104 is closed. Thus, the outlet port 269 of the venturi device 26, which is connected to the second inlet port 102, is itself isolated, that is to say that the gas mixture containing 40 ppmv of NO present in the internal volume 260a of the main body 260 cannot pass through the outlet port 269. Thus, the entirety of the gas flow rate, called the useful flow rate, which is equal to the sum of the flow rate leaving the calibrated orifice 25 by its outlet diameter 25a and the flow rate of air entering through the intake orifice 263, will escape to the ambient atmosphere A via the exhaust conduit 265.

Once the manual valve 23 is in the open position, and preferably upon confirmation of this open position by the user via the GUI, the control means 15 control the solenoid valve 104 to fluidically connect the second upstream port 104b to the downstream port 104c. Thus, the second line 103 becomes fluidically connected to the gas analysis line 110.

Because of the connection of the calibration device 2 of the invention to the second inlet port 102 of the NO delivery apparatus 1, the internal volume 260a of the main body 260 is then in fluidic relationship, via the outlet port 269 of the venturi device 26, to gas analysis line 110.

The pump 140, which is still controlled to take a flow rate of the order of 250 ml/min, will then aspirate some of the useful flow rate (i.e. sum of the flow rate leaving the calibrated orifice 25 by its outlet diameter 25a and of the flow rate entering through the venturi intake orifice 263), thus exposing the measuring means 120-122, namely the NO2, NO and O2 sensors, to a gas mixture containing 40 ppmv of NO.

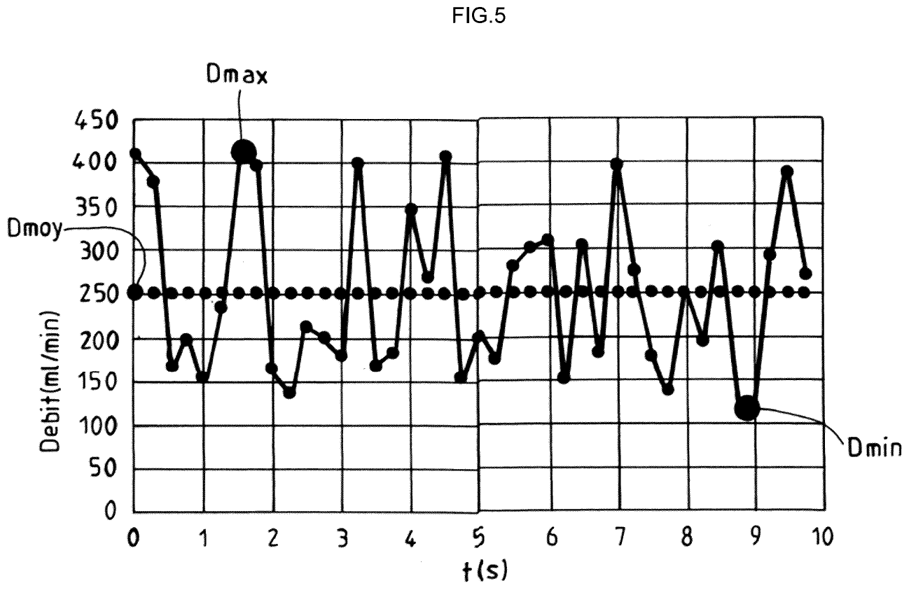

FIG. 5 shows schematically the standard performance of a diaphragm pump used as gas aspiration device 140 within the gas analysis line 110 of the NO delivery apparatus 1 of FIG. 4.

More precisely, the curve of flow rate as a function of time in FIG. 5 shows that there are rapid fluctuations in the flow rate passing through the gas analysis line 110, which are measured by the flow rate sensor 130.

If the mean flow rate value Dmoy, for example calculated over 10 seconds, is, as expected, approximately 250 ml/min, the instantaneous flow rate oscillates between a maximum flow rate value Dmax of approximately 400 ml/min and a minimum flow rate value Dmin of approximately 125 ml/min.

This requires the venturi device 26 to deliver a useful flow rate greater than the maximum flow rate value Dmax measured by the flow rate sensor 130. Indeed, if this useful flow rate is less than the maximum flow rate Dmax, then any flow rate requested by the pump 140 that is greater than the useful flow rate will be completed by the admission of ambient air A via the exhaust conduit 265 of the venturi device 26. However, this undesired admission via the exhaust conduit 265 will then result in a dilution of the mixture present in the internal volume 260a of the main body 260 of the venturi device 26, and therefore in a reduction in the desired NO concentration (which should be equal to 40 ppmv here).

Referring to FIG. 2, it will be noted that the useful flow rate, which is the sum of the flow rate leaving the calibrated orifice 25 through its outlet diameter 25a and the flow rate entering through the venturi intake orifice 263, is 0.73 l/min (730 ml/min) for a useful expansion pressure of 350 mb, and 1.06 l/min (1060 ml/min) for a useful expansion pressure of 750 mb. By setting the precision regulator to 500 mb, the useful flow rate will be much higher than the maximum flow rate Dmax requested by the pump.

In other words, the sum of the flow rate leaving the calibrated orifice 25 through its outlet diameter 25a and the flow rate entering through the venturi intake orifice 263 will always be greater than the instantaneous demand of the pump 140, and therefore the excess gas, i.e. excess mixture, will follow the exhaust conduit 265 of the venturi element 26 so as to be discharged to the ambient atmosphere.

It follows that the gas passing through the outlet port 269 of the venturi device 26, the second inlet port 102, the second line 103 and the gas analysis line 110 has the desired NO concentration, namely here 40 ppmv.

This mixture at the desired concentration will then expose the NO sensor 121 to the target concentration.

After a stabilization phase, the control means 15 can determine the high calibration point of the NO sensor 121 at 40 ppmv.

The control means 15 can then stop the pump 140 so that no more gas circulates in the gas analysis line 110. The measuring means 120, 121, 122 are consequently exposed to a gas with an initial concentration equal to 40 ppmv of NO and furthermore comprising an O2 content of the order of 20.9% vol.

It is known that, in the presence of oxygen, NO progressively oxidizes to NO2, in particular as a function of their respective contents and their contact time.

By way of an established model, in the form of an equation relating an NO2 concentration to a given time as a function of the initial NO and O2 contents, the control means 15 allow a reaction of conversion of the NO to NO2 to take place during a given time, for example for 8 minutes, until a given NO2 content is formed, for example until 5 ppmv NO2 is formed. This NO2 content then serves as the high calibration point of the NO2 sensor 120.

The measuring means 120-122, in particular the NO2 sensor 120, NO sensor 121 and O2 sensor 122, are then perfectly calibrated.

The control means 15 can then initiate a final step consisting in controlling the solenoid valve 104 so as to fluidically connect its first upstream port 104a to its downstream port 104c and to control the pump 140 to regulate a flow rate, measured by the flow rate sensor 130, of the order of 250 ml/min, as described above, so as to circulate ambient air A in the gas analysis line 110 for “purging” it of the gases, in particular the NO, that may be present therein.

At the same time, the control means 15 can preferably inform the user that the calibration procedure has been carried out, so as to prompt him to close the manual valve 23 in order to stop the generation of a gas mixture at 40 ppmv by the venturi device 26.

The calibration device 2 of the invention can then be detached from the second socket 102 of the NO delivery apparatus 1 and stored pending a new calibration procedure.

The calibration device 2 of the invention makes it possible to simplify the procedure of regular calibration of the sensors of the gas analyser 10 of the NO delivery apparatus 1, in particular the NO sensor, the NO2 sensor and the O2 sensor.

What is claimed is:

1. Calibration device (2) for calibrating the measuring means (120-122) of an NO delivery apparatus (1), said measuring means (120-122) comprising an NO sensor, an NO2 sensor and an O2 sensor, said calibration device (2) comprising a gas intake line (22) comprising, arranged in series:
a valve (23) for controlling the circulation of gas in the gas intake line (22),
a precision regulator (24) configured to deliver a predefined fixed pressure in the gas intake line (22),
a calibrated orifice device (25), and
a venturi device (26) comprising a main body (260) defining an internal volume (260a) and comprising:
a gas inlet orifice (265a) fluidically connected to the calibrated orifice device (25) in order to allow gas from the calibrated orifice device (25) to enter the internal volume (260a),
a gas intake orifice (263) fluidically connected to the atmosphere (A) in order to allow air to enter the internal volume (260a),
an exhaust orifice (265a) fluidically connected to the atmosphere (A), for discharging some of the gas contained in the internal volume (260a) to the atmosphere (A), and
an outlet port (269) for supplying at least some of the gas contained in the internal volume (260a) to an NO delivery apparatus (1).

2. Device according to claim 1, characterized in that the valve (23) is movable between at least:
a closed position, in which the circulation of gas in the gas intake line (22) is prevented, and
an open position, in which the circulation of gas in the gas intake line (22) is permitted.

3. Device according to claim 1, characterized in that the precision regulator (24) is configured to deliver a fixed pressure of between 350 and 700 mbar.

4. Device according to claim 1, characterized in that the calibrated orifice device (25) comprises an outlet orifice (25a) with a diameter of less than 500 μm.

5. Device according to claim 1, characterized in that the gas intake orifice (263) has a cross-sectional area of between 0.5 and 10 mm2.

6. Device according to claim 1, characterized in that the gas intake line (22), the valve (23), the precision regulator (24), the calibrated orifice device (25) and the venturi device (26) are arranged in a common housing (200).

7. Device according to claim 1, characterized in that the valve (23) is a manual valve actuatable by a user.

8. Device according to claim 1, characterized in that it further comprises upstream connection means (20) arranged at an inlet port (21) of the gas intake line (22) and configured to connect a flexible conduit (33) thereto.

9. Device according to claim 1, characterized in that it further comprises downstream connection means (269a) arranged at the outlet port (269) of the venturi device (26) and configured to connect thereto a gas feed line (103) of a gas analyser (10) of an NO delivery apparatus (1).

10. Device according to claim 1, characterized in that the outlet diameter of the calibrated orifice (25) and the diameter of the venturi intake orifice (263) are dimensioned to have a constant ratio, between the flow rate leaving the calibrated orifice (25) and the flow rate entering through the venturi intake orifice (263), of between 10 and 30.

11. Use of a calibration device (2) according to claim 1, for calibrating the measuring means (120-122) of an NO delivery apparatus (1), said measuring means (120-122) comprising an NO sensor, an NO2 sensor and an O2 sensor.

12. Use according to claim 11, characterized in that the calibration device (2) is fluidically connected to a gas cylinder (3, 31) containing an NO/N2 mixture feeding the gas intake line (22) of the calibration device (2), upstream of the valve (23).
